(19) United States
(12) Patent Application Publication
(10) Pub. No.: US 2012/0150215 A1
(43) Pub. Date: Jun. 14, 2012

(54) Junctional Bleed Device

(75) Inventor: Mark Louis Donald, Norfolk, VA (US)

(73) Assignee: Juarez Industries, Norfolk, VA (US)

(21) Appl. No.: 13/313,486

(22) Filed: Dec. 7, 2011

Related U.S. Application Data
(60) Provisional application No. 61/421,248, filed on Dec. 9, 2010.

(51) Int. Cl. A61B 17/00

(52) U.S. Cl. 606/203

(57) ABSTRACT

Systems and methods of applying a bandage to a patient including securing a bandage device over a wound and around a body part of the patient. The bandage device includes a platform with a plurality of first openings for use with a tensioning mechanism, and at least one second opening for use with a pressure adjusting mechanism. The pressure adjusting mechanism may include threads, detents, and other adjustable, or semi-permanent, locking mechanisms. In addition to pressure applied to a wound via the tensioning mechanism, an additional pressure may be applied to the wound through the platform of the bandage device via the pressure adjusting mechanism. The pressure adjusting mechanism may have a pressure application mechanism that applies the additional pressure to the wound.

### CROSS-REFERENCE TO RELATED APPLICATIONS

This application claims priority to and benefit under 35 U.S.C. §119(e) to U.S. Provisional Application No. 61/421,248, filed Dec. 9, 2010, the disclosure of which is herein expressly incorporated by reference in its entirety.

### BACKGROUND OF THE INVENTION

The invention relates generally to articles of manufacture including bandages, and also finds applicability in methods of applying bandages. Aspects of the invention are particularly well suited for use in bandages applied to “junctional” areas of the body meaning major joint areas such as the shoulder, axillary (aka underarm), and the hip, pelvis and groin.

A bandage is a piece of material used typically to support a dressing, a splint, and/or a part of a patient's body. Bandages are available in a wide range of types, that may perform different functions and be particularly suited for different applications. For example, common gauze bandages may be made of a light cotton, or other gauze material, and be used to hold a dressing in place, whereas a compression bandage may be made from combinations of materials that provide light to moderate compression when applied adhesive to the skin of, and/or wrapping around, a body part of the patient.

Different types of bandages are more or less suited to particular applications based on such factors as the material, size, and intended use of the bandage. One limitation common with most conventional bandages is the inability to apply increased pressure to a wound site after the bandage is secured. Additionally, conventional bandages are limited in their ability to apply pressure to certain areas of the body in which recesses, and other changes in curvature, reduce the coefficient of pressure compared to the tension of the bandage. These problem can be particularly acute in situations with puncture, and other traumatic, wounds in the areas of, for example, the axillary, shoulder, pelvis, hip and/or groin, where major arteries may be compromised without effective tourniquet options.

In light of the above, and other problems with conventional bandages known in the art, there is an ongoing need for more effective bandage devices and techniques.

The invention provides articles of manufacture, and methods of using bandages, including a junctional bleed device as described herein.

### SUMMARY OF THE INVENTION

According to aspects of the invention, a platform for use with a bandaging device is provided by which an additional pressure may be applied to a wound, beyond pressure applied through the tensioning of the bandage.

Embodiments may include a bandaging device with a platform. In embodiments, the platform may include a plurality of first openings configured to receive a tensioning mechanism therethrough. In embodiments, the platform may include at least one second opening configured to receive a pressure application mechanism. The at least one second opening may be configured to oppose an additional pressure applied through the platform to a wound.

In embodiments, at least one of the plurality of first openings may include a securing mechanism configured to fixedly secure a strap of the tensioning mechanism in a plurality of positions.

In embodiments, the at least one second opening may include female threads for receiving the pressure application mechanism, and the platform may be configured to increase the additional pressure by turning a male-threaded shaft in the at least one second opening.

In embodiments, the at least one second opening may include a detent for receiving a protrusion of the pressure application mechanism, and the platform may be configured to increase the additional pressure by advancing a shaft of the pressure application mechanism through the platform. The detent may be configured to substantially prevent the shaft from withdrawing through the platform after the shaft is advanced.

In embodiments, the at least one second opening may include a release mechanism for quickly reducing and/or releasing the additional pressure. For example, an exemplary releasing mechanism may be configured to release the shaft from the detent, or the like, and thereby allow the shaft to withdraw through the platform.

In embodiments, the platform may be rigid. The platform may constructed from at least one of a rigid nylon, plastic, composite, and metal material. In embodiments, the platform may be configured to be flexible in a first direction. The platform may be manufactured from a substantially transparent material.

According to other aspects of the invention, a bandaging device may include a tensioning mechanism for use with the platform. Exemplary tensioning mechanisms may include, for example, bandage wraps, nylon straps, webbing, belts, and the like. Bandage wraps of the tensioning device may include an elastic wrap, and/or may be made of woven fabrics containing elastomeric filaments interspersed among non-elastomeric filler yarns, elastic non-woven fabrics of synthetic fibers, and other materials and combinations of materials known in the art. In other embodiments, the bandage wrap of the tensioning device may be a substantially inelastic material, such as nylon and/or spun fiber yarns such as cotton, viscose, polyester, cotton/viscose or cotton/polyester mixtures and other materials and combinations of materials known in the art.

In embodiments, the tensioning mechanism may be fixed to at least one of the plurality of first openings in the platform. In embodiments, the tensioning mechanism may include at least one free end that is adjusts a length of the tensioning device through at least one of the plurality of first openings in the platform.

According to other aspects of the invention, a bandaging device may include a pressure adjusting mechanism for use with the platform. The pressure adjusting mechanism may be configured to apply an additional pressure through the platform to a wound. For example, the pressure application mechanism may include a shaft with male threads for engaging female threads of an opening in the platform, and the bandaging device may be configured to increase the additional pressure by turning the male-threaded shaft. In embodiments, the pressure application mechanism may include a shaft with at least one of a protrusion and/or recess for engaging with a detent, or the like, in an opening of the platform. The bandaging device may be configured to increase the additional pressure by advancing the shaft through the platform, the detent, or the like, substantially preventing the shaft from withdrawing through the platform via engagement with the at least one of a protrusion and/or recess after the shaft is advanced.

In embodiments, the bandaging device may include a pressure application mechanism for use with the pressure adjusting mechanism. For example, a pressure application mechanism may be permanently, or irremovably, affixed to a working end of the pressure adjusting mechanism that is applied to the wound area. The pressure application mechanism may include a pad that is configured to conform, at least partially to the area of the wound. For example, the pad may be constructed from a soft rubber material, and the like, that may substantially conforms to a curved surface of the body, such as depressions in the shoulder, axillary, groin and pelvic regions, under pressure.

In embodiments, the pressure application mechanism may be sized for a particular application, e.g. use in the area of the shoulder, axillary, groin, pelvis, hip, inner-elbow, inner-knee, etc. The pressure application mechanism may include an attachment mechanism, such as screw threads, quick-release attachments, and the like, for attaching to the pressure adjusting mechanism. In embodiments, a bandaging device kit may include a plurality of differently configured, sized and/or shaped pressure application mechanisms, e.g. for use with different types of wounds.

In embodiments, the pressure application mechanism may include a core material, e.g. at least partially within the pad, that is configured to resist deformation. For example, the core may be constructed from a hard rubber, nylon, and or plastic material, and the like, that substantially resists deformation under working pressure. The core may be used to transmit the pressure necessary for the pad to substantially conform to a curved surface of the body, such as depressions in the shoulder, axillary, groin and pelvic regions.

According to yet further aspects of the invention, methods of applying a bandage to a patient may include securing a bandage device over a wound and around a body part of the patient. The bandage device may include a platform as described herein, and may be used to apply an additional pressure to the wound through the platform of the bandage device.

In embodiments, the additional pressure may be adjustable. For example, an adjustable amount of additional pressure may be applied by turning a threaded shaft, advancing a lockable shaft, and the like, through a platform of the bandaging device. According to embodiments, the additional pressure may be applied to, for example, a shoulder area, an axillary area, a groin area, a pelvic area, and/or an extremity, such as the inside of the elbow or knee, of the patient.

Embodiments may also include adjusting a tension of the bandaging device via at least one first opening in the platform through which an end of the tensioning mechanism passes. For example, the first opening may include a securing mechanism to substantially prevent a bandage wrap, nylon strap, etc, from withdrawing. In other embodiments, the tensioning mechanism may include its own securing device, such as hooks, loops, buckles, etc. that adjustably secure the tensioning mechanism to itself.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention claimed. The detailed description and the specific examples, however, indicate only preferred embodiments of the invention. Various changes and modifications within the spirit and scope of the invention will become apparent to those skilled in the art from this detailed description.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced. In the drawings:

FIG. 1 depicts an isometric view of an exemplary bandaging device according to principles of the invention.

FIGS. 2A-2C depict three views of an exemplary platform according to principles of the invention.

FIGS. 3A and 3B depict an exemplary platform with a tensioning mechanism according to principles of the invention.

FIG. 4 depicts a side view, and a partial cut-away view, of an exemplary bandaging device according to principles of the invention.

FIGS. 5A and 5B depict side views, and a partial cut-away view, of an exemplary bandaging device according to principles of the invention.

FIG. 6 depicts various locations on a human body where bandaging devices and methods according to the invention may be used.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a lesion” is a reference to one or more lesions and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

The term “bandage” as used herein refers to pieces of material used typically to protect, immobilize, compress, or support a wound or injured body part, and/or support a dressing, a splint, or similar medical device. Bandages are distinct from, but may by packaged with or attached to, dressings, that are typically applied directly to a wound. Bandages may be elastic or inelastic.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” individuals afflicted with conditions requiring bandaging, as the term “treating” is used herein, encompasses treatment of conditions that suggest the applicability of bandages, with or without splints, dressings and other medical devices typically secured with bandages, to the symptomatic individual.

The term “patient” as in treatment of “a patient” refers to a mammalian individual afflicted with or prone to a condition, disease or disorder as specified herein, and includes both humans and animals.

The following preferred embodiments may be described in the context of exemplary bandage packs for ease of description and understanding. However, the invention is not limited to the specifically described bandage packs and methods, and may be adapted to various clinical applications without departing from the overall scope of the invention.

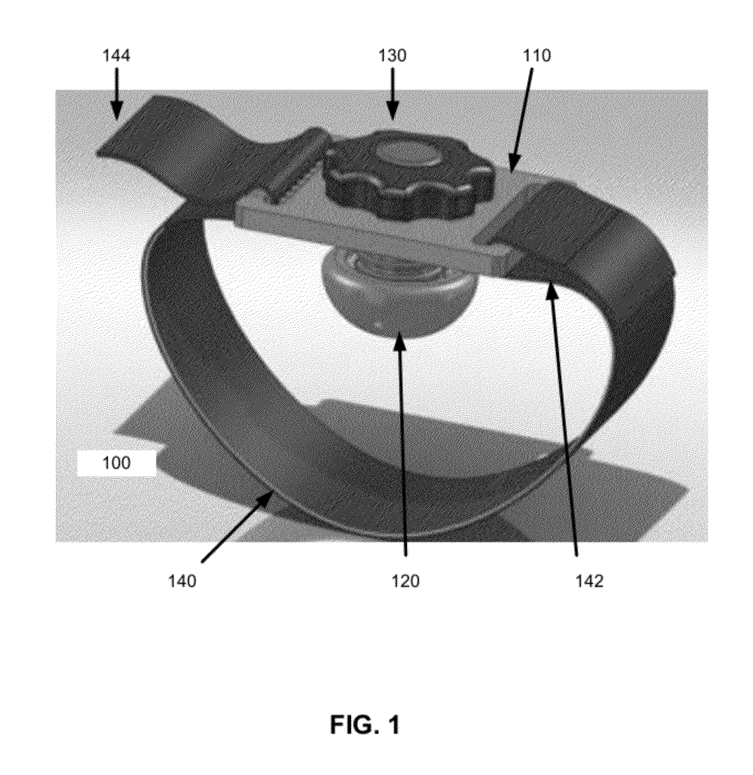

FIG. 1 depicts aspects of an exemplary bandaging device 100 according to principles of the invention. As shown in FIG. 1, bandaging device 100 may include exemplary platform 110, pressure pad 120, screw assembly 130, and strap 140.

Strap 140 includes a fixed end 142 secured to the platform 110, and a free end 144, that may be run through and adjustably secured with respect to platform 110. Thus, in use, the bandaging device 100 may be secured around a body part of the patient, such as the joint areas of the shoulder, axillary, pelvis, hip, groin, elbow, knee, etc., by placing the pressure pad 120 over the wound and wrapping the strap 140 around the body part. Strap 140 may be used as an exemplary tensioning mechanism and may include, for example, bandage wraps, nylon straps, webbing, belts, and the like. Bandage wraps of the tensioning device may include an elastic wrap, and/or may be made of woven fabrics containing elastomeric filaments interspersed among non-elastomeric filler yarns, elastic non-woven fabrics of synthetic fibers, and other materials and combinations of materials known in the art. In other embodiments, the bandage wrap of the tensioning device may be a substantially inelastic material, such as nylon and/or spun fiber yarns such as cotton, viscose, polyester, cotton/viscose or cotton/polyester mixtures and other materials and combinations of materials known in the art.

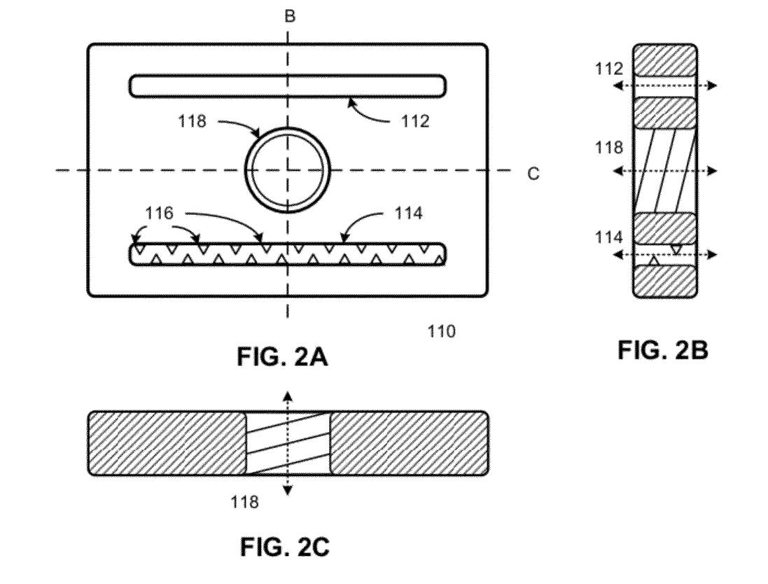

Tension may be applied to the bandaging device 100 by pulling the free end 144 and securing it to the strap 140 and/or fixing it in an opening of the platform 110. The tension may contribute to a first pressure on the wound via the pressure pad 120, which is typically applied over a dressing (not shown). An additional pressure may also be applied to the wound, in a more direct manner, by turning the screw assembly 130 to advance a shaft of the screw assembly through the platform 110, thus advancing the pressure pad 120 toward the wound. Further details of an exemplary platform 110 are shown in FIGS. 2A-2C.

As shown in FIG. 2A, a platform 110 may include a plurality of first openings 112, 114 for receiving the strap 140, or other tensioning mechanisms, therethrough. In embodiments, first openings 112, 114 may also be produced, for example, with an open end for easily inserting and/or removing a strap from the side of the platform. More than two first openings may also be included, in various orientations, for example, to increase the number of tensioning directions that may be applied, such as by using multiple straps with the platform.

One of the first openings 114, is shown with additional restraining teeth 116 for adjustably securing the strap 140 in the opening 114. Restraining teeth 116 may be configured, for example, to resist movement of the strap 140 through the platform unless the strap 140 is oriented in a certain way, e.g. perpendicular, or some other angle, with respect to the platform 110. Restraining teeth 116 may be configured with a release mechanism (not shown) that releases the teeth 116 from engagement with strap 140 when activated. Any such lockable, releasable, adjustable, and/or “one-way”, mechanisms known in the art for securing a strap through a buckle, and the like, may be used.

As also shown in FIG. 2A, a platform 110 may include a second opening 118 that is configured to receive a pressure adjusting mechanism, such as screw assembly 130. For example, second opening 118 may include female screw threads corresponding to male threads of a screw assembly 130 shaft. Thus, the platform 110 may be used to resist an additional pressure applied to a wound as the screw assembly 130 is advanced through the platform 110. Second opening 118 may also include a release mechanism (not shown) that may quickly release engagement with the screw assembly 130.

In embodiments, the platform 110 may be rigid. The platform 110 may constructed from at least one of a rigid nylon, plastic, composite, and metal material. In embodiments, the platform 110 may be configured to be flexible in a first direction, such as in the direction of dashed line B, to allow the platform to at least partially conform around a body part, such as the leg. The platform 110 may be manufactured from a substantially transparent material, such as plastic, which may, for example, allow improved visibility to the wound area and dressing underneath the bandaging device. FIGS. 2B and 2C show cutaway views along dashed lines B and C, respectively.

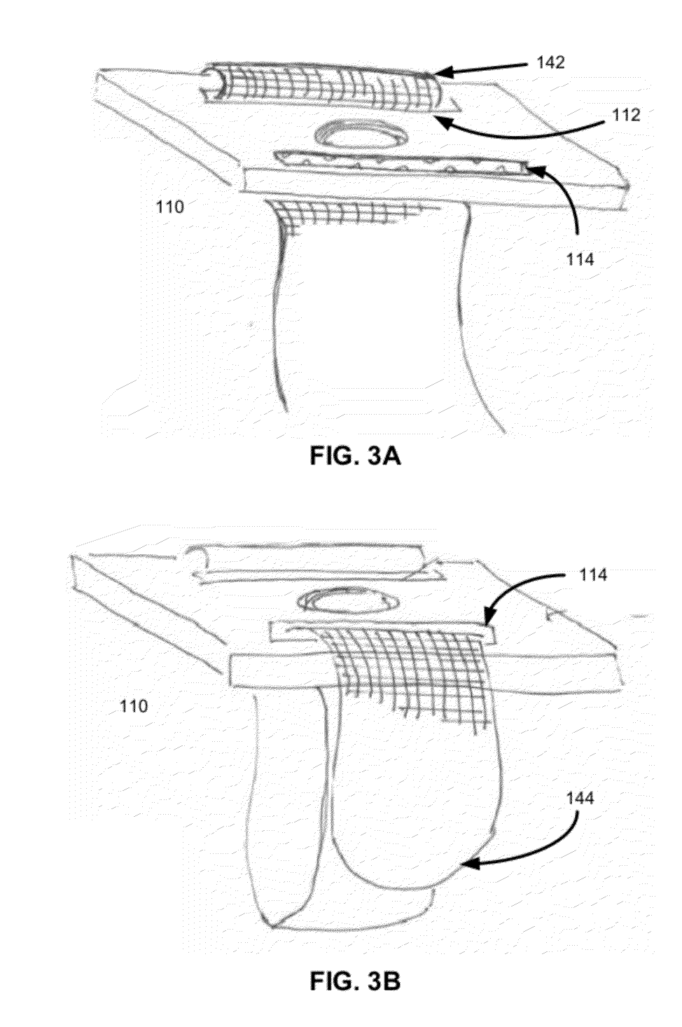

FIGS. 3A and 3B show additional details with respect to the application of a platform 110 to a wound area. As show in FIG. 3A, a platform 110 may be assembled with an end 142 of strap 140 affixed to the platform 110, e.g. through a first opening 112. The strap 140 may then be wrapped around a body part (not shown) and the free end 144 drawn through opening 114, as shown in FIG. 3B. As mentioned above, opening 114 may have its own securing mechanism contained therein, or proximate thereto, and/or strap 140 may have securing mechanism(s) such as, for example, hooks, loops, buckles, and other means known in the art.

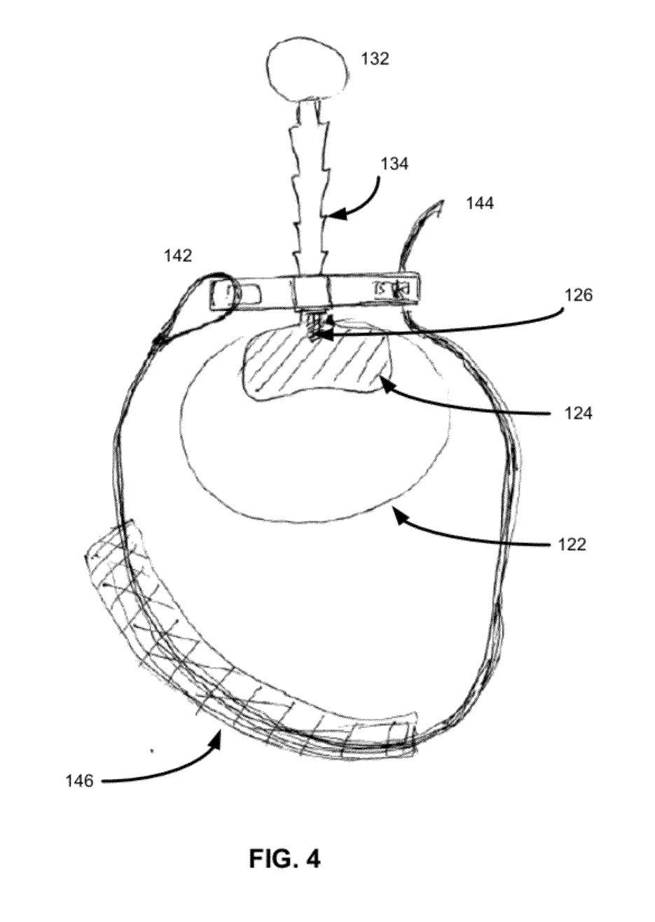

FIG. 4 depicts an alternative embodiment, and partial cutaway view, of an exemplary bandaging device including a pad 122, a core 124, an attachment mechanism 126, shaft 134 and knob 132. As shown in FIG. 4, rather than a screw assembly, a pressure adjusting mechanism may include a shaft 134 with ridges, indentations, and the like, for engaging with detents, and the like, in a second opening of the platform. Thus, the shaft 134 may be advanced through the platform by applying direct pressure to knob 132, rather than turning a screw assembly as described with respect to FIG. 1. Detents, and the like, in the second opening of the platform may substantially prevent the shaft 134 from withdrawing. In embodiments, a release mechanism may be provided to disengage the detents, and the like, from the shaft, allowing the shaft top withdraw through the platform.

As also shown in the partial cutaway view of FIG. 4, a pad 122 may partially surround a core 124. In embodiments, the pad 122 may be configured to conform, at least partially to the area of a wound. For example, the pad 122 may be constructed from a soft rubber material, and the like, that may substantially conform under pressure to a curved surface of the body, such as depressions in the shoulder, axillary, groin and pelvic regions.

In embodiments, the pad 122 may be sized and/or shaped for a particular application, e.g. use in the area of the shoulder, axillary, groin, pelvis, hip, inner-elbow, inner-knee, etc. Various shapes may include, for example, spherical, ellipsoid, cylindrical, trapezoidal, conical, etc. In embodiments, a bandaging device kit may include a plurality of differently configured, sized and/or shaped pressure application mechanisms, e.g. with different shaped pads 122 and/or cores 124, different sized pads 122 and/or cores 124, different material pads 122 and/or cores 124, etc., for use with different types/locations of wounds. In embodiments, the pad 122 may be at least partially inflatable, such as by an external pressure source or integral pump (not shown) in the crank 136 or knob 132. Embodiments may also include a bladder (not shown) inflatable by similar means. Inflatable pressure mechanisms may be advantageous, for example, in better conforming the pad 122 to depressions, and the like, in the patient's anatomy.

In embodiments, a core material of the core 124 may be configured to resist deformation. For example, the core 124 may be constructed from a hard rubber, nylon, and/or plastic material, and the like, that substantially resists deformation under working pressure. The core 124 may be used to transmit the pressure necessary for the pad 122 to substantially conform to a curved surface of the body, such as depressions and/or bumps in the shoulder, axillary, groin and pelvic regions.

In embodiments, the attachment mechanism 126 may be used to attach and/or remove the assembly including the core 126 and pad 122 from the bandaging device. The attachment mechanism 126 may include various attachment features such as, for example, screw threads, quick-release attachments, and the like. Alternatively, the assembly including the core 126 and pad 122 may be permanently attached to the shaft 134, and the like. In embodiments, the attachment mechanism 126, and/or other permanent attachment point may include a swivel for rotating the pad 122 about the axis of the shaft 134. Such configurations may be advantageous, for example, when the pad 122 is irregularly shaped, such as an ellipsoid, that can better conform to areas of the body, such as a groin or axillary of the patient.

As also shown in FIG. 4, the alternative embodiment may also include a strap with a fixed end 142 and a free end 144, and a sleeve 146. The sleeve 146 may be used, for example, to ease positioning of the bandaging device and/or distribute the pressure applied by the strap to the body part that it is wrapped around. It should be understood that, although shown with a fixed end 142, exemplary bandaging devices may be provided without a fixed end strap, such as by providing a platform without an integral strap. Such configurations may allow for further flexibility in the types of tensioning devices used with the device, such as using a belt of the patient, or other expedient means for wrapping the device around a body part of the patient.

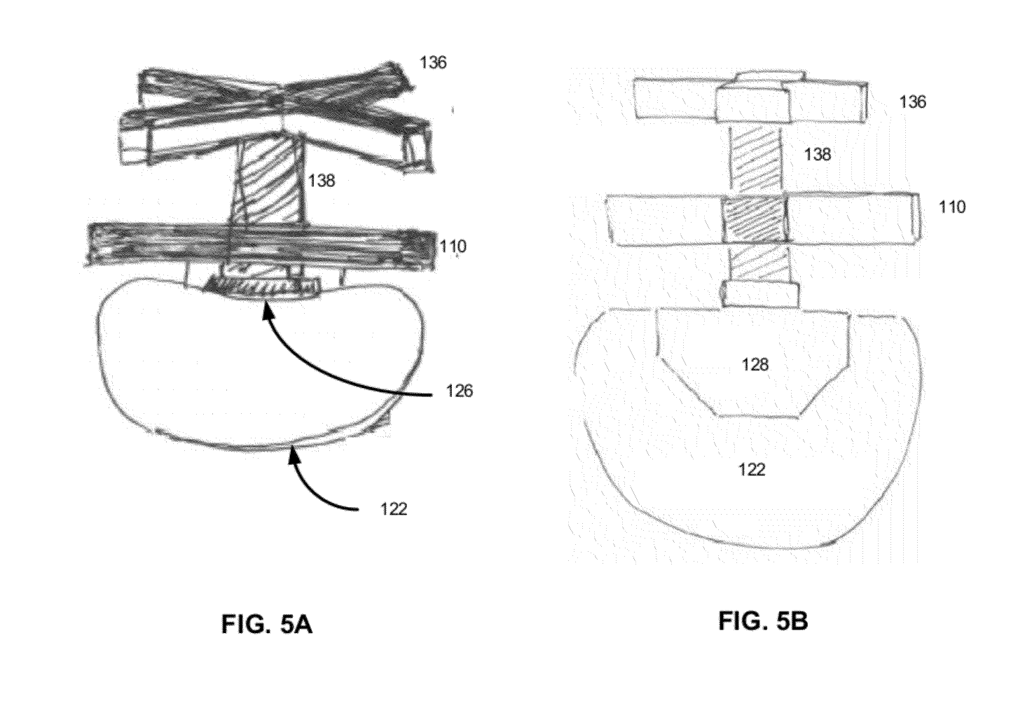

FIGS. 5A and 5B depict further details regarding application of additional pressure using a bandaging device, such as bandaging device 100 shown in FIG. 1. As shown in FIG. 5A, a bandaging device including a pad 122, attachment mechanism 126, platform 110, threaded shaft 138 and crank 136 may be configured in a first state in which the shaft 138 is substantially withdrawn with respect to platform 110. A user may turn crank 136, thereby turning shaft 138, to advance the shaft 138 to a second state with respect to the platform 110, as shown in FIG. 5B. In the second state shown in FIG. 5B, the pad 122 receives additional pressure via shaft 138 and core 128, such that the pad 122 may be able to substantially conform to an area of the wound, and/or apply additional pressure to a wound site, through opposition with the platform 110.

As shown in the partial cutaway of the pad 122 and core 128 shown in the FIG. 5B, the core 128 may be shaped to distribute a force applied via shaft 138 in a distributed and/or non-linear manner. For example, core 128 may have a plurality of angled surfaces, as shown in FIG. 5B, curved surfaces, and the like, to apply force to pad 122 in a variety of directions and magnitudes, including force vectors with outward components.

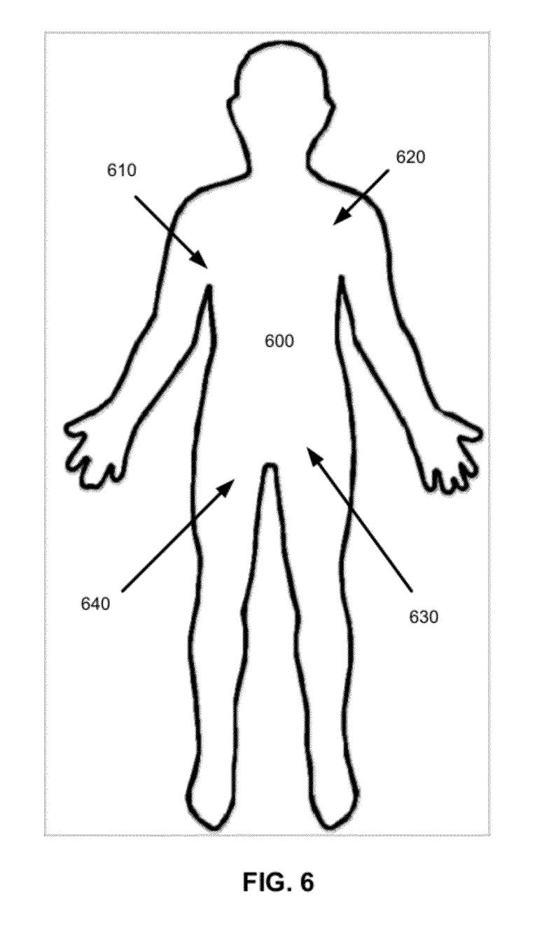

As described herein, the bandaging device and methods of the invention may find particular applicability in the application to “junctional wounds” including wounds to the shoulder, axillary, groin and pelvic regions, as well as other contoured areas such as the insides of the elbows and knees. FIG. 6 shows some of the areas to which the devices and methods may be applied, e.g. the shoulder 620, axillary 610, groin 640 and pelvis 630. In embodiments, methods of applying a bandage to a patient may include securing a bandage device over a wound and around a body part of the patient. For example, in the case of applying the bandage to a wound in the groin area 640, the device may be secured around the leg and/or parts of the buttocks of the patient. In other embodiments, such as when the bandage is applied to a wound in the shoulder area 620, the device may be secured around the arm and/or parts of the back and chest of the patient. In embodiments, a plurality of straps may be used, for example, to provide additional tensioning directions to the platform of the device. Additional straps may be provided, for example, across a back, chest, pelvis, hip, etc. of the patient. In other embodiments where the device is applied to a wound in the inner elbow or knee, the device may be secured around the arm or leg of the patient and the pad of the device applied to the wound area.

As described herein, an additional pressure provided though a platform of the device may be adjustable. For example, an adjustable amount of additional pressure may be applied by turning a threaded shaft, advancing a lockable shaft, and the like, through a platform of the bandaging device. According to embodiments, the additional pressure may be applied to, for example, a shoulder area 620, an axillary area 610, a groin area 640, a pelvic area 630, and/or an extremity, such as the inside of the elbow or knee, of the patient. In embodiments, a pressure pad of the device may be configured to substantially conform under the additional pressure to a recess of the patient's body, and a user may apply such additional pressure until a suitable shape is obtained.

Embodiments may also include adjusting a tension of the bandaging device via at least one first opening in the platform through which an end of the tensioning mechanism passes. For example, the first opening may include a securing mechanism to substantially prevent a bandage wrap, nylon strap, etc, from withdrawing. In other embodiments, the tensioning mechanism may include its own securing device, such as hooks, loops, buckles, etc. that adjustably secure the tensioning mechanism to itself.

The description given above is merely illustrative and is not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments.

What is claimed is:

1. A bandaging device comprising:
a platform;
a tensioning mechanism;
a pressure adjusting mechanism; and
a pressure application mechanism,
wherein, the pressure adjusting mechanism is configured to apply an adjustable amount of pressure through the platform.

2. The bandaging device of claim 1, wherein the platform includes a plurality of first openings configured to receive the tensioning mechanism therethrough.

3. The bandaging device of claim 2, wherein at least one of the first openings includes a securing mechanism configured to fixedly secure a strap of the tensioning mechanism.

4. The bandaging device of claim 1, wherein the platform includes at least one second opening through which the pressure adjusting mechanism is received.

5. The bandaging device of claim 4, wherein:
the at least one second opening includes female threads for receiving the pressure application mechanism;
the pressure application mechanism includes a shaft with male threads for engaging the female threads of the at least one second opening; and
the bandaging device is configured to increase the adjustable amount of pressure by turning the shaft.

6. The bandaging device of claim 4, wherein:
the at least one second opening includes a detent for receiving a protrusion of the pressure application mechanism;
the pressure application mechanism includes a shaft with the protrusion; and
the bandaging device is configured to increase the adjustable amount of pressure by advancing the shaft through the platform, the detent substantially preventing the shaft from withdrawing through the platform after the shaft is advanced.

7. The bandaging device of claim 6, further comprising a releasing mechanism configured to release the protrusion from the detent, and thereby allow the shaft to withdraw through the platform.

8. A platform for use with a bandaging device, comprising:
a plurality of first openings configured to receive a tensioning mechanism at least partially therethrough; and
at least one second opening configured to receive a pressure application mechanism and to oppose an additional pressure applied through the platform to a wound.

9. The platform of claim 8, wherein at least one of the plurality of first openings includes a securing mechanism configured to fixedly secure a strap of the tensioning mechanism.

10. The platform of claim 8, wherein:
the at least one second opening includes female threads for receiving the pressure application mechanism; and
the platform is configured to increase the additional pressure by turning a male-threaded shaft in the at least one second opening.

11. The platform of claim 8, wherein:
the at least one second opening includes a detent for receiving a protrusion of the pressure application mechanism; and
the platform is configured to increase the additional pressure by advancing a shaft of the pressure application mechanism through the platform, the detent substantially preventing the shaft from withdrawing through the platform after the shaft is advanced.

12. The platform of claim 11, further comprising a releasing mechanism configured to release the shaft from the detent, and thereby allow the shaft to withdraw through the platform.

13. The platform of claim 8, wherein the platform is rigid.

14. The platform of claim 13, wherein the platform is constructed from at least one of a rigid nylon, plastic, composite, and metal material.

15. The platform of claim 8, wherein the platform is flexible in a first direction.

16. A method of applying a bandage to a patient comprising:
securing a bandage device over a wound and around a body part of the patient, the bandage device including a platform;
applying an additional pressure to the wound through the platform of the bandage device.

17. The method of claim 16, wherein the additional pressure is adjustable.

18. The method of claim 16, wherein the additional pressure is applied to a shoulder area of the patient.

19. The method of claim 16, wherein the additional pressure is applied to an axillary area of the patient.

20. The method of claim 16, wherein the additional pressure is applied to a groin area of the patient.

21. The method of claim 16, wherein the additional pressure is applied to a pelvic area of the patient.
